(19) United States
(12) Patent Application Publication
(10) Pub. No.: US 2015/0369806 A1
(43) Pub. Date: Dec. 24, 2015

(54) DETECTION OF VIRAL DISEASES USING A BIOCHIP THAT CONTAINS GOLD NANOPARTICLES

(71) Applicants: National Chung Hsing University, Taichung (TW); Academia Sinica, Taipei (TW)

(72) Inventors: GOU-JEN WANG, Taichung (TW); Shie-Liang Hsieh, Taipei (TW); Yen-Ting Tung, Kaohsiung (TW)

(21) Appl. No.: 14/742,305

(22) Filed: Jun. 17, 2015

Related U.S. Application Data
(60) Provisional application No. 62/014,509, filed on Jun. 19, 2014.

(30) Foreign Application Priority Data
Aug. 12, 2014 (TW) 103127672

(51) Int. Cl. G01N 33/569; G01N 33/543; C25D 3/48; C23C 14/34; C23C 14/18; G01N 33/553; G01N 27/12

(52) U.S. Cl. CPC G01N 33/56983; G01N 33/553; G01N 33/54346; G01N 33/54353; G01N 33/54366; G01N 27/12; C23C 14/34; C23C 14/18; C25D 3/48; G01N 2333/185

(57) ABSTRACT

A sensor for detecting molecular interactions between a target and a binding domain by electrochemical impedance spectroscopy. The sensor includes an anodic aluminum oxide barrier layer having a gold-coated array of regularly spaced nano-hemispheres and gold nanoparticles coated with a binding domain attached thereto. Also provided are methods for producing the sensor and for using the sensor to detect the presence of a virus in a sample.

The current application claims a foreign priority to an application in Taiwan by application number 103127672, filed on Aug. 12, 2014, and a priority to U.S. 62/014,509, filed on Jun. 19, 2014.

### BACKGROUND

1. Field of the Invention

The invention relates to sensors for detecting interactions between a target, such as a virus, and a binding domain using electrochemical impedance spectroscopy.

2. Background Information

Dengue virus (DV) is one of the most common mosquito-borne viral diseases in humans with 50-100 million cases being recorded annually. Infection with DV causes a range of clinical symptoms including dengue fever and dengue hemorrhagic fever.

C-type lectins on macrophages or dendritic cells have been shown to play a critical role in DV infection. Both the mannose receptor and the Dendritic Cell-Specific Intercellular adhesion molecule-3-Grabbing Non-integrin (DC-SIGN) receptor have been reported to regulate DV binding and entry, while C-type lectin domain family 5, member A (CLEC5A) mediates DV-induced proinflammatory cytokine production and pathogenesis. The interaction between CLEC5A and DV is weak compared to the interaction between DC-SIGN and DV. Specific interactions between CLEC5A and DV can only be measured over a narrow range of concentration by an innate immunity receptor enzyme-linked immunoassay.

Electrochemical impedance spectroscopy (EIS), which is sensitive to the conjugation between a receptor and its substrate through the changes of the impedance at an electrode-solution interface, has been used for the detection of the strong binding between DV and an antibody. Of note, the antibody-DV binding is much stronger than the glycan-mediated interaction between DV and CLEC5A.

Typical assays for detecting viruses in a sample, including ELISA and surface plasmon resonance, rely on the immobilization of a detection probe on a flat surface. The sensitivity of flat surface-based assays is limited, as the immobilized probe, e.g., an antibody or a receptor-Fc fusion protein, typically extends from the surface by only 5-12 nm. As a result, the contact area between a three-dimensional particle, e.g., a virus, and the probe is limited to that between a small fraction of the particle surface and the limited number of probes capable of contacting it.

The need exists for a sensor which can effectively detect weak interactions between the binding domain such as glycoproteins and their receptors and, alternatively, can detect small amounts of a target using a high-affinity probe.

### SUMMARY

To satisfy the need mentioned above, the present invention provides a sensor for detecting molecular interactions between a target and a binding domain by electrochemical impedance spectroscopy.

The sensor includes the following components: (i) a substrate, (ii) an anodic aluminum oxide (AAO) barrier layer attached to the substrate, (iii) an Au coating of 10-50 nm affixed to the AAO barrier layer, (iv) a plurality of gold nanoparticles (GNP), each having a diameter of 2-10 nm, deposited on the Au coating, and (v) a binding domain attached to each GNP.

The AAO barrier layer includes an array of regularly spaced nano-hemispheres spaced apart by 5-30 nm, each nano-hemisphere having a diameter of 30-300 nm.

Also, the present invention provides a method for producing an electrochemical impedance spectroscopy sensor for detecting molecular interactions.

In one embodiment, the method includes the steps of forming an anodic aluminum oxide (AAO) barrier layer having an array of regularly spaced nano-hemispheres spaced apart by 5-30 nm, each nano-hemisphere having a diameter of 30-300 nm, attaching the AAO barrier layer to a substrate, coating the AAO barrier layer with Au, attaching gold nanoparticles (GNPs) having a diameter of 210 nm to the Au-coated AAO barrier layer to form a nanostructured surface, activating the nanostructured surface, and attaching a binding domain to the activated nanostructured surface, thereby forming a sensor for detecting molecular interactions.

Additionally, the present invention provides a method is provided for detecting a virus in a sample.

In one embodiment, the method includes the steps of providing the sensor described above containing a binding domain that specifically binds to the virus, measuring the charge transfer resistance of the sensor, contacting the sensor with a sample, and again measuring the charge transfer resistance. The second charge transfer resistance is greater than the first charge transfer resistance if the virus is present in the sample.

According to the present invention, it can catch the target by binding with at least one binding domain and keep the target in the restricting space. In other words, the target is hard to away from the sensor.

### BRIEF DESCRIPTION OF THE DRAWINGS

The invention description below refers to the accompanying drawings, of which:

FIG. 1A is a partial view of an embodiment of the invention.

FIG. 1B is a top view of the FIG. 1A.

FIG. 1C is a side view of the FIG. 1A.

FIG. 1D is a cross-sectional view along the cross line D-D of the FIG. 1A.

FIG. 2A is a schematic diagram of the sensor of the invention contacting with the target.

FIG. 2B is an enlarged partial view of the circle part labeled “B” of the FIG. 2A.

FIG. 3A is shown the surface of the AAO barrier-layer without the GNPs in a electron microscope.

FIG. 3B is shown the surface of the AAO barrier-layer with the GNPs in a electron microscope.

FIG. 4A is the equivalent circuit model used for calculating charge transfer resistance of a sensor;

FIG. 4B is a Nyquist plot of charge transfer resistance of a sensor including a CLEC5A binding domain before and after addition of DV;

FIG. 5 is a bar graph of the change in impedance of a sensor after binding of DV to a sensor surface coated with the indicated binding domains;

FIG. 6 is a plot of the change in impedance of a sensor after binding of DV versus DV concentration; and

FIG. 7 is a bar graph of the change in impedance of a sensor after binding of mutant and wild-type viruses to a sensor surface coated with the indicated binding domains.

### DETAILED DESCRIPTION

The details of one or more embodiments of the invention are set forth in the description below. Other features, objects, and advantages of the invention will be apparent from the description, the drawings, the appendix, and the claims. Although the following detailed description contains many specifics for purposes of illustration, a person of ordinary skill in the art will appreciate that many variations and alterations to the following details are within the scope of the invention. Therefore, the following embodiments of the invention are set forth without any loss of generality to, and without imposing limitations upon, the claimed invention.

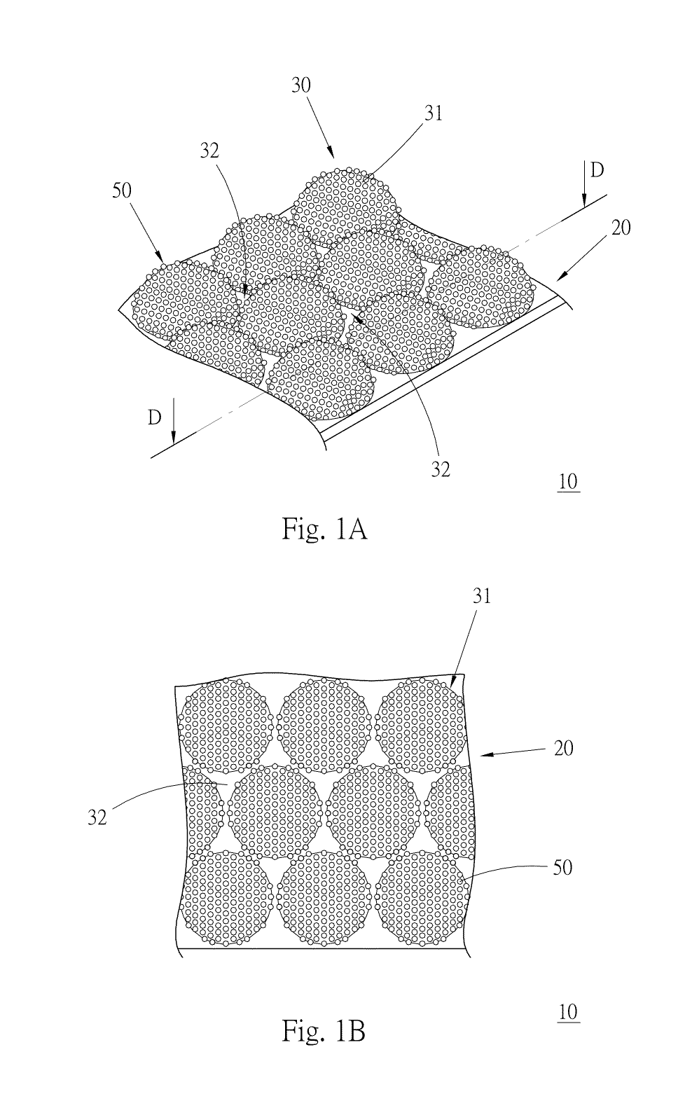

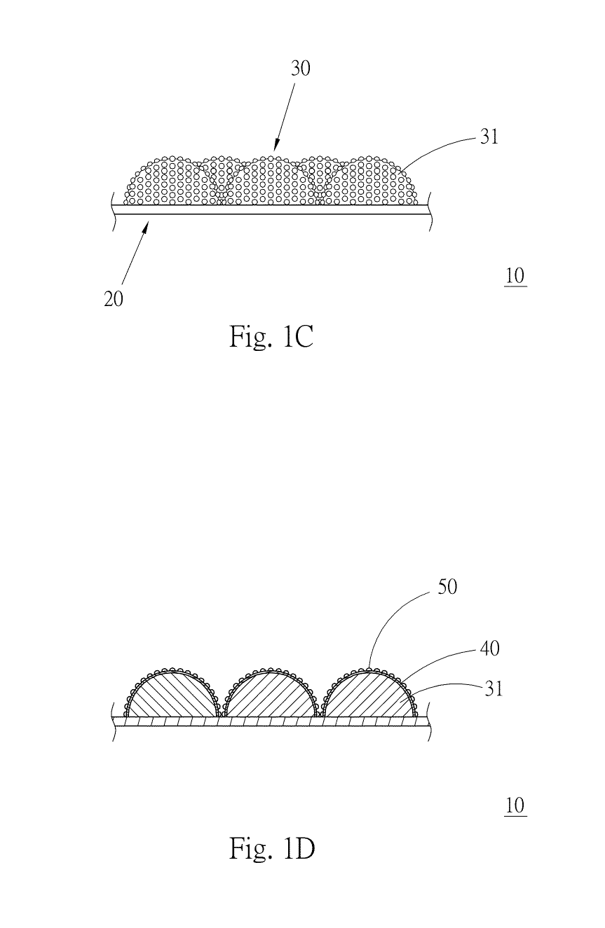

As mentioned above, please see the FIGS. 1A to 1D, 2A and 2B, a sensor for detecting molecular interactions between a target and a binding domain by electrochemical impedance spectroscopy 10 includes a substrate 20, an anodic aluminum oxide (AAO) barrier layer 30, an Au coating 40, a plurality of gold nanoparticles (GNP) 50 and a binding domain 60.

The AAO barrier layer 30 which is attached to the substrate 20 having an array of regularly spaced nano-hemispheres 31. The nano-hemispheres 31 can be spaced apart by 5-30 nm, e.g., 5, 10, 15, 20, 25, and 30 nm.

Each nano-hemisphere 31 can have a diameter of 30-300 nm, e.g., 30, 40, 50, 75, 100, 150, 200, 250, and 300 nm diameter.

The dimensions for nano-hemisphere 31 spacing and diameter can be selected depending upon the desired target. For example, if the target is a large virus, a sensor having a larger diameter and a wider spacing of the nano-hemispheres 31 can be constructed.

The Au coating 40 is used for modifying the AAO barrier layer 30 by coating it with a 10-50 nm (e.g., 10, 20, 30, 40, and 50 nm) thin film coating of Au. The Au coating 40 serves both as an electrode for GNP 50 deposition and as the working electrode during operation of the sensor 10.

The gold nanoparticles (GNPs) 50 are deposited on the Au coating 40. The GNPs 50 have a diameter of 2-10 nm, e.g., 2, 3, 4, 5, 6, 7, 8, 9, and 10 nm. The density of GNPs 50 deposited on the Au coating 40 can be from 2500 to 6250 GNPs per μm2.

In a particular embodiment, the sensor 10 includes a conductor (not shown in figures) in electrical contact with the Au coating 40. The conductor serves to electrically connect the Au coating 40 to a potentiostat to facilitate deposition of the GNPs 50 during assembly of the sensor 10. The conductor can also serve to electrically connect the Au layer to a potentiostat during operation of the sensor 10.

The above-described modified AAO barrier layer is attached to a substrate. The substrate 20 can be glass or a corrosion-resistant polymer. In a specific embodiment, the substrate 20 is glass.

The binding domain 60 attached to each GNP 50. In a specific embodiment, the binding domain 60 can be covalently attached to each GNP 50. The binding domain can be an antibody, a receptor, a recombinant protein, a glycolipid, a lectin, or a glycan.

In an embodiment, the binding domain 60 is a receptor-Fc fusion protein. The receptor-Fc fusion protein can include a glycan-binding domain. For example, the receptor-Fc fusion protein can include the glycan binding domain of CLEC5A, DC-SIGN (dendritic-cell-specific intercellular adhesion molecule-3-grabbing non-integrin), and Dectin-2. In a preferred embodiment, the receptor-Fc fusion protein includes a glycan-binding domain of CLEC5A.

In another embodiment, the binding domain 60 is a lectin that specifically binds to a mannose residue, a fucose residue, a sialic acid residue, a glucosamine residue, or a galactosamine residue.

The binding domain 60 is selected depending upon the desired target to be detected by the sensor. For example, the binding domain 60 can bind specifically to a eukaryotic cell or cell fragment, a virus, a bacterium, a peptide, and a recombinant protein. Preferably, the binding domain 60 specifically binds to a molecule located on the surface of a cell or a virus. In a particular embodiment, the binding domain 60 is multi-valent. For example, the multi-valent binding domain 60 can specifically bind to at least two molecules simultaneously on the surface of the target cell or virus. Similarly, the multi-valent binding domain 60 can specifically bind to the same molecule on the surface of two or more target cells or viruses simultaneously.

In certain embodiments, the binding domain 60 can bind specifically to a herpesvirus, an adenovirus, a parvovirus, a papilloma virus, a poliovirus, an influenza virus, a rotavirus, a flavivirus, or a poxvirus. In a particular embodiment, the binding domain specifically binds to hepatitis B virus, human immunodeficiency virus, hepatitis C virus, H5N1 influenza virus, SARS virus, Japanese encephalitis virus (JEV), eastern equine encephalitis virus, West Nile virus, yellow fever virus, mumps virus, lymphocytic choriomeningitis virus, coronavirus, or Dengue virus.

Binding of a binding domain to a target is considered to be specific binding if the change in impedance of a sensor upon binding of the target to the binding domain is at least 30% greater than the change in impedance of a negative control binding domain (i.e., a binding domain known not to bind to the target) upon incubation with the target.

As discussed above, the binding domain 60 can be a glycan. In certain embodiments, the binding domain 60 is a glycan that includes a terminal sialic acid residue in an a 2-3 linkage or in an a 2-6 linkage. Sensors including such glycans can be used to detect the presence of an influenza virus in a sample via binding of influenza hemagglutinin. Advantageously, sensors including these glycans can be used to determine the host range of the influenza virus. More specifically, if binding of the influenza virus to the a 2-3-linked sialic acid is detected and binding to the a 2-6-linked sialic acid is not, the influenza virus can infect humans but not avian species. Similarly, an influenza virus capable of binding to a 2-6-linked sialic acid and not a 2-3-linked sialic acid will infect avian species and not humans.

In a particular embodiment, the binding domain 60 is Globo H, stage-specific embryonic antigen 3 (SSEA3), or stage-specific embryonic antigen 4 (SSEA4). Globo H, SSEA3, and SSEA4 are carbohydrate antigens found predominantly on the surface of cancer cells. Antibodies against these antigens, e.g., anti-Globo H antibodies, are often elicited in an individual if cancer cells are present. A sensor including one or more of these binding domains 60 can be used to detect in a sample low levels of anti-Globo H antibodies. Such a sensor can also detect low-affinity anti-Globo H antibodies. The presence of anti-Globo H antibodies in a subject indicates that the subject has cancer. The cancer can be, but is not limited to, breast cancer, prostate cancer, and lung cancer.

In another embodiment, please see the FIGS. 2A and 2B, the nano-hemispheres 31 are arranged in staggered manners to form a restricting space 32 among the three adjacent nano-hemispheres 31. While contacting the sensor 10 with a sample, a target 70 of the sample will be restricted in the restricting space 32, and bind with the binding domains 60 of the three nano-hemispheres 31 around the restricting space 32 simultaneously. By the restricting space 32, the target 70 will be binding with at least one binding domain 60 and hardly separate from the sensor 10, so it can strengthen the molecular interactions between the target 70 and the binding domains 60, and promote the detecting accuracy and sensitivity.

As set forth supra, a method is provided for producing the electrochemical impedance spectroscopy sensor for detecting molecular interactions. First, an AAO barrier layer having an array of regularly spaced nano-hemispheres is formed. The nano-hemispheres can be spaced apart by 5-30 nm, e.g., 5, 10, 15, 20, 25, and 30 nm. Each nano-hemisphere can have a diameter of 30-300 nm, e.g., 30, 40, 50, 75, 100, 150, 200, 250, and 300 nm diameter. These dimensions can be controlled by adjusting the process parameters such as the etchant, applied potential, and current.

In a preferable embodiment, the nano-hemispheres are arranged in staggered manners to form a restricting space among the three adjacent nano-hemispheres.

Forming the AAO barrier layer can include a step of removing non-oxidized aluminum beneath the barrier layer. The non-oxidized aluminum can be removed, e.g., by treating it with CuCl2/HCl, leaving behind only the AAO barrier layer.

The AAO barrier layer is coated with a thin Au film. As mentioned above, the Au film can be 10-50 nm (e.g., 10, 20, 30, 40, and 50 nm). The Au thin film can be deposited on the AAO barrier layer by sputtering. The sputtering can be direct current sputtering, radio frequency sputtering, or radio frequency magnetron sputtering.

The Au thin film is coated with GNPs having a diameter of 2-10 nm (e.g., 2, 3, 4, 5, 6, 7, 8, 9, and 10 nm). The coating can be accomplished by electrochemical deposition. In a particular embodiment, the GNPs are deposited using 0.5 mM HAuCl4 as the working electrolyte and applying a DC −0.7 V electric potential for 3 minutes.

The AAO barrier layer including the Au thin film and GNPs, termed the nanostructured surface, is attached to a substrate. As mentioned above, the substrate can be glass. In an embodiment, the AAO barrier layer is assembled onto a glass slide using an epoxy. The entire assembly is sealed such that, during use, a working buffer cannot leak into the sensor. In an embodiment, the assembly is sealed using silica gel.

In order to construct a sensor to detect a desired target, a binding domain which binds specifically to the target is attached to the surface of the just-described nanostructured surface. The binding domains attached are described in detail above. The attachment can be via covalent or non-covalent bonding to the nanostructured surface. In a preferred embodiment, a binding domain can be attached to the nanostructured surface using a self-assembling monolayer (SAM) process.

Also within the scope of the invention is a method for detecting a virus in a sample. First, a sensor is provided containing a binding domain that specifically binds to the virus. Examples of binding domains that can be used and viruses that can be detected are set out above. The sensor is then blocked to reduce the background signal. The sensor can be blocked with the same medium in which the virus is suspended, minus the virus. For example, the sensor can be blocked by incubating it in culture medium for 45 min. Optimum blocking conditions can be determined by a person of ordinary skill in the art.

The charge transfer resistance of the blocked sensor is measured using a potentiostat. The measurements are performed using a counter electrode, reference electrode, and working electrode. The counter electrode can be a Pt film, the reference electrode can be Ag/AgCl, and the working electrode is the sensor surface. The measurements can be performed in a PBS buffer that contains a mixing electrolyte of 5 mM Fe(CN)₆⁴⁻ and 5 mM Fe(CN)₆³⁻.

The blocked sensor, after measuring its charge transfer resistance, is incubated with a sample containing the target, e.g., a virus. For example, the blocked sensor can be incubated with a sample containing a virus for 15-60 min. In a particular embodiment, the blocked sensor is incubated with a sample containing a virus for 30 min. The charge transfer resistance is again measured after the incubation period and compared to that measured prior to incubation.

In a particular embodiment, the sample is a tissue sample or a blood sample. In a preferable embodiment, the sensor includes CLEC5A as the binding domain and the target is a flavivirus, e.g., DV and JEV.

The method and sensor described above are advantageous for detecting weak binding between a binding domain and a target. The binding of a lectin to a sugar, e.g., a sugar on a glycan, is typically 10-100 fold weaker than that between an antibody and a protein antigen. The sensor described supra including a lectin as binding domain, e.g., CLEC5A, can readily detect weak binding of a glycan antigen, such as the envelope protein of DV.

Alternatively, a high affinity antibody against a desired target can be included in the sensor as the binding domain. Such a sensor can be 100-fold more sensitive than an ELISA assay using the same antibody. More specifically, an antibody which, in an ELISA, is capable of detecting a lower limit of a target on the order of hundreds of picograms can be used as a binding domain in the sensor to detect 1-10 picograms of a target. Thus, a sensor including a high-affinity anti-HIV antibody as binding domain has the potential to detect the low levels of HIV present during the clinical latency period of an HIV infection.

The sensor described above is also compatible with existing sandwich ELISA methods utilizing an antibody to detect a target captured by the binding domain on the sensor.

Without further elaboration, it is believed that the above description has adequately enabled the present invention. The following examples are, therefore, to be construed as merely illustrative, and not limitative of the remainder of the disclosure in any way whatsoever. All documents cited herein are incorporated by reference in their entirety.

### EXAMPLE 1

### Production of a Nano-Structured Sensor

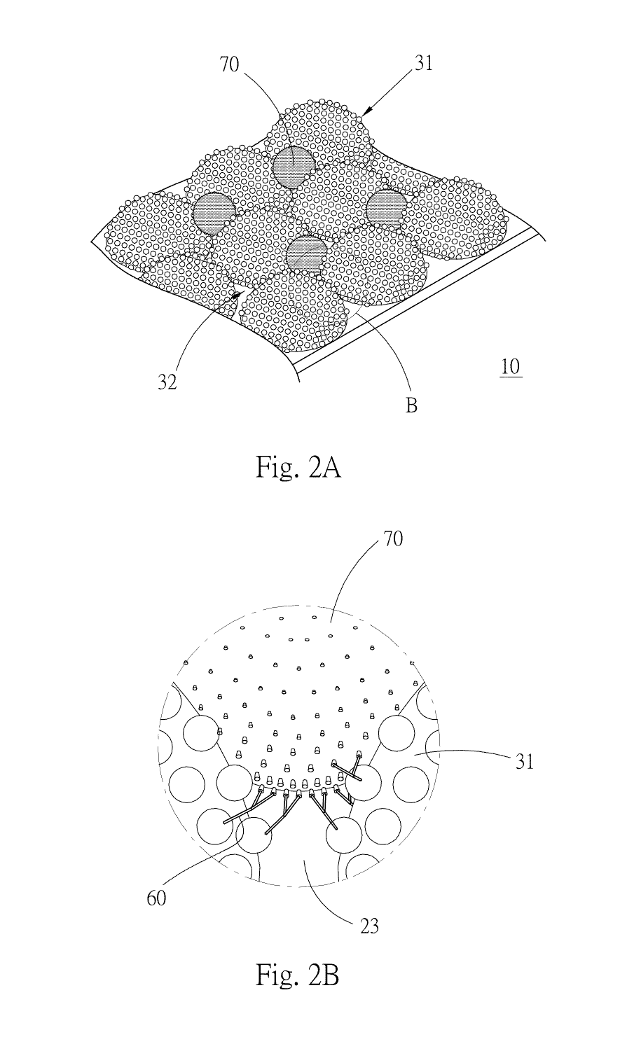

To increase the surface reacting area as compared to a flat surface, the barrier layer surface of an AAO membrane was employed as the surface of the sensor chip. The AAO membrane was prepared by the anodization process described in a previous study (Tsai et al. Int. J. Nanomedicine 6:1201-1208). Following anodic oxidization, the non-oxidized aluminum beneath the barrier layer was removed by incubating it in a CuCl₂/HCl solution that was prepared by dissolving 13.45 g of CuCl₂ powder in 100 ml of a 35 wt % hydrochloric acid solution. This results in formation of a barrier layer having evenly distributed nano-hemispheres (see FIG. 3A). The nano-hemisphere structure of the barrier layer was further modified by incubating it in 30 wt % phosphoric acid for 30 minutes. A 10 nm gold thin film was sputtered onto the surface of the AAO barrier-layer via DC sputtering to form an electrode for further electrochemical deposition of GNPs (see FIG. 3B).

The consistency of the working area was assured by gluing onto the prepared AAO barrier layer a piece of plastic paraffin film (PARAFILM®) with a 6 mm diameter hole. An SP-150 potentiostat (Bio-Logic, USA) was used to conduct the electrochemical deposition of GNPs. The gold thin film covered sample was placed at the working electrode, with the gold thin film serving as the electrode. The counter electrode was a Pt film and the reference electrode was Ag/AgCl. GNPs were uniformly deposited on the nano-hemisphere surfaces using 0.5 mM HAuCl₄ as the working electrolyte by applying a DC −0.7 V electric potential for 3 minutes. The entire sensor was sealed with silica gel to prevent EIS working buffer from leaking into the sensor.

### EXAMPLE 2

### Preparation of Binding Domains

Binding domains including a human IgG1 Fc region and the Fc region fused at its Fab region to lectin ligands (CLEC5A, DC-SIGN, or Dectin-2), were constructed as previously described (Chen et al., Nature 453:672-676). Recombinant proteins were overexpressed in a FREESTYLE™ 293 Expression System (Invitrogen). In brief, 3×10⁷ 293-F cells were transfected with a mixture of 40 μl of 293FECTIN™ reagent and 30 μg of binding domain constructs. At days 3 and 5 after transfection, culture supernatants were collected. The recombinant binding domains were further purified from the supernatants using protein A beads.

### EXAMPLE 3

### Immobilization of Binding Domains to the Sensor Surface

The self-assembling monolayer (SAM) process was applied to immobilize the binding domains onto the GNPs which were deposited on the sensor surface as described in EXAMPLE 1 above. The electrode surface was treated with 20 μL of 10 mM 11-mercaptoundecanoic acid (11-MUA) for 10 minutes, followed by incubation with 20 μL of a mixture of 50 mM N-hydroxysuccinimide (NHS) and 100 mM 1-ethyl-3-(3-dimethylaminopropyl)-carbodiimide (EDC). The binding domains (0.02 μg/μL for control hIgG1 and 0.012 μg/μL for other binding domains in a volume of 15 μL) were then incubated with the treated surface for 30 minutes to link them to the sensor surface.

### EXAMPLE 4

### Preparation of Virus Stocks

The propagation of DV and JEV were performed in C6/36 cells using standard methods. Enterovirus EV71(BrCr strain; ATCC VR784) was propagated in Vero cells at 37° C. as described previously (Shih et al., Antimicrob. Agents Chemother. 48:3523-3529). Viral titers were measured by plaque-forming-assays in BHK-21 cells.

### EXAMPLE 5

### Electrical Impedance Spectroscopy Detection and Quantification

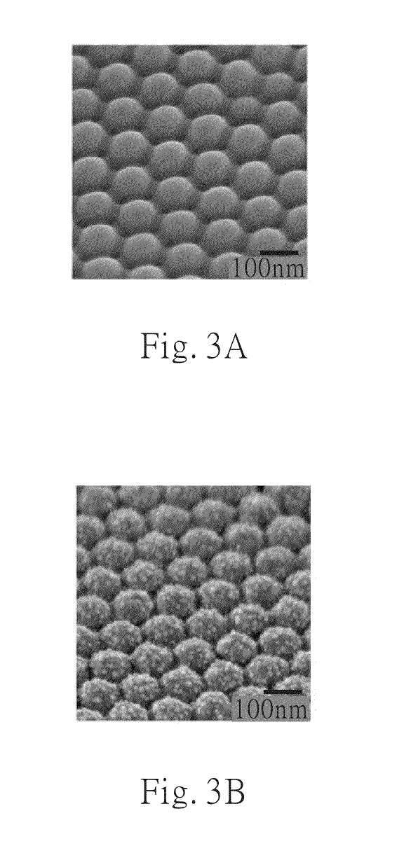

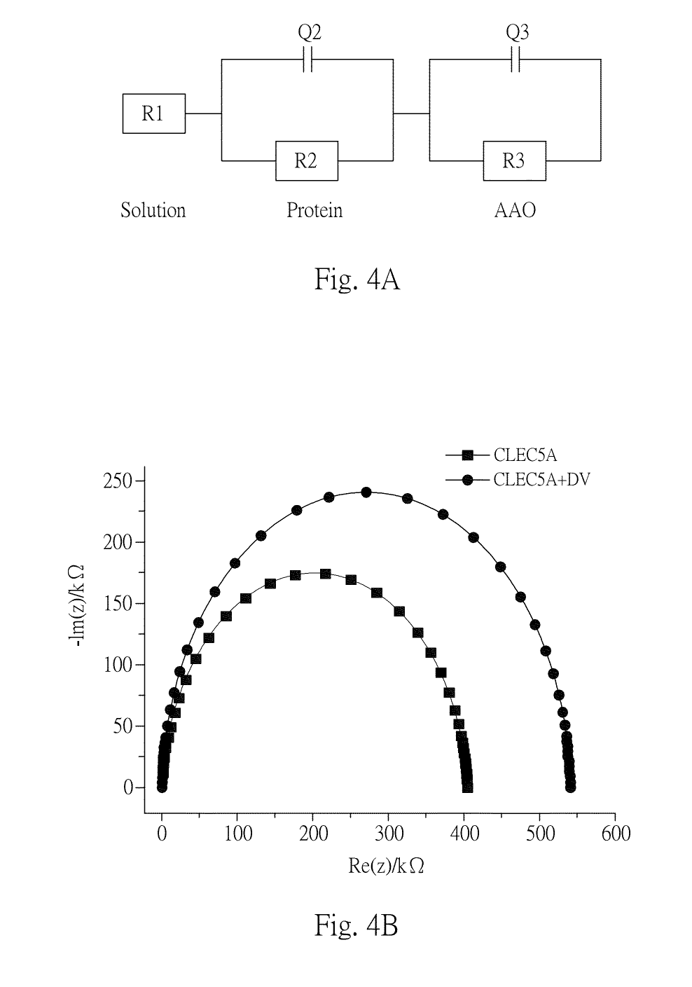

The sensors described above were blocked with culture medium for 45 min. The charge transfer resistance of the blocked sensor was measured over a frequency range of 0.1 Hz to 100 kHz using an SP-150 potentiostat. The sensors were then incubated with 9.5×10⁷ plaque forming units/mL (pfu/mL) of DV, JEV, or EV for 30 min. The charge transfer resistance was measured again after the incubation of the sensor with DV, JEV, or EV. All measurements were performed in a PBS buffer solution that included a mixing electrolyte of 5 mM Fe(CN)₆⁴⁻ and 5 mM Fe(CN)₆³⁻. The counter electrode, reference electrode, and working electrode were Pt film, Ag/AgCl, and the sensor surface, respectively. The data thus obtained was fitted into an equivalent circuit model (see FIG. 4A) to calculate the charge transfer resistance. Values were plotted on a Nyquist plot (see FIG. 4B), wherein in the FIG. 4A, “R1” is the resistance of the electrolyte, “Q2” is the capacitance of the probe, “R2” is the resistance of the probe, “Q3” is the capacitance of the AAO barrier-layer and “R3” is the resistance of the AAO barrier-layer.

### EXAMPLE 6

### Detecting Binding Between CLEC5A and DV

Sensors as described above were prepared with each one of the binding domains, CLEC5A, DC-SIGN, Dectin-2, and hIgG1. DC-SIGN, which interacts strongly with glycans on the envelope protein of DV, was used as a positive control to demonstrate the normal ligand-receptor interaction. As the engineered binding domains described above have an IgG-like structure, the Dectin-2 receptor, which recognizes α-mannans in the cell wall of fungi, was used as an isotype control to control for any effects of the IgG-like structure. The human IgG1 served as the negative control for determining the background. EV served as a negative control to detect non-specific binding of virus to hIgG1.

The differences in the charge transfer resistance (AR) between the sensor before and after addition of virus test samples was calculated and expressed as mean±SEM. The statistical significance of the difference between data gathered for CLEC5A versus the control binding domains was determined using Student's t-test. Differences were considered as significant at a two-tailed P value of <0.05. The results are shown in FIG. 3A.

The ΔR for the sensor bearing the CLEC5A binding domain and incubated with DV (n=7) was significantly higher (p<0.05) than the corresponding value in the isotype control sensor (Dectin-2+DV, n=5) and negative control sensor (hIgG1+DV, n=7; hIgG1+EV, n=3). Clearly, the weak interaction between CLEC5A and DV, which is barely detected by ELISA, can be clearly and readily detected with the sensor described herein.

### EXAMPLE 7

### CLEC5A-DV interactions Under Different Viral Concentration

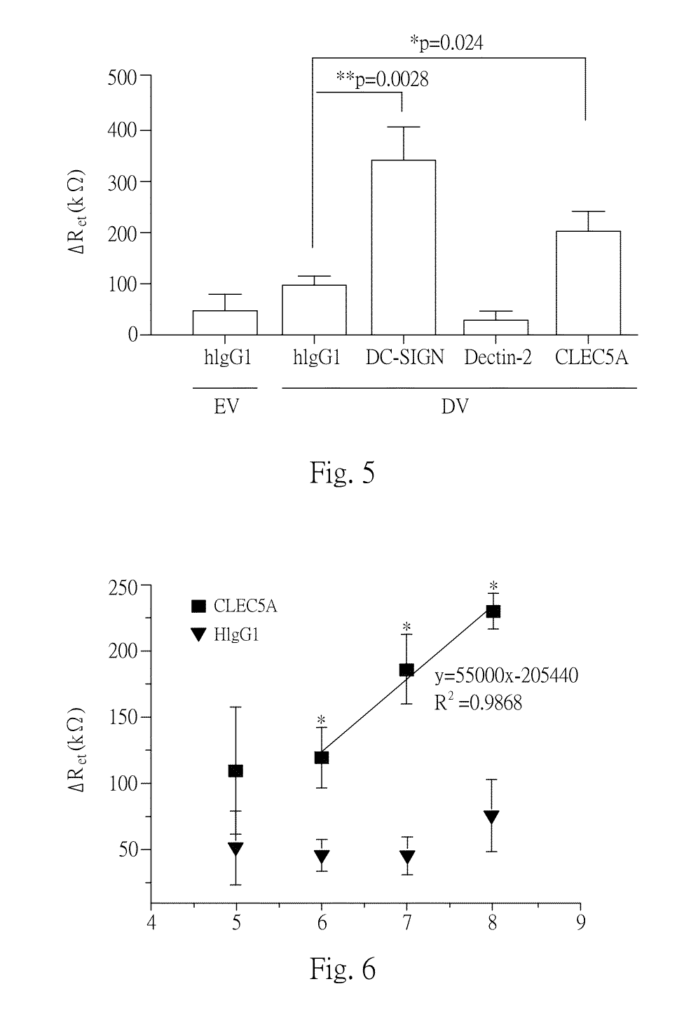

Serial dilutions of DV from 9.5×10⁷ to 9.5×10⁴ pfu/mL were used to determine the detection limits of a sensor device containing a CLEC5A binding domain. The results are shown in FIG. 6. A significant increase of charge transfer resistances were found for a virus titer between 9.5×10⁷ and 9.5×10⁵ pfu/mL (p<0.05) as compared to a sensor bearing the hIgG1 negative control. A linear relationship between ΔR and the log of virus titer was evident (R²=0.9868) for DV titers between 10⁸ and 10⁶ pfu/mL. See FIG. 6. The sensor including a CLEC5A binding domain allows for the accurate determination of virus titer based on the variations of the charge transfer resistance within the range of 169 kΩ to 224 kΩ.

### EXAMPLE 8

### Detection of Differences in Glycosylation of Viral Envelope Proteins

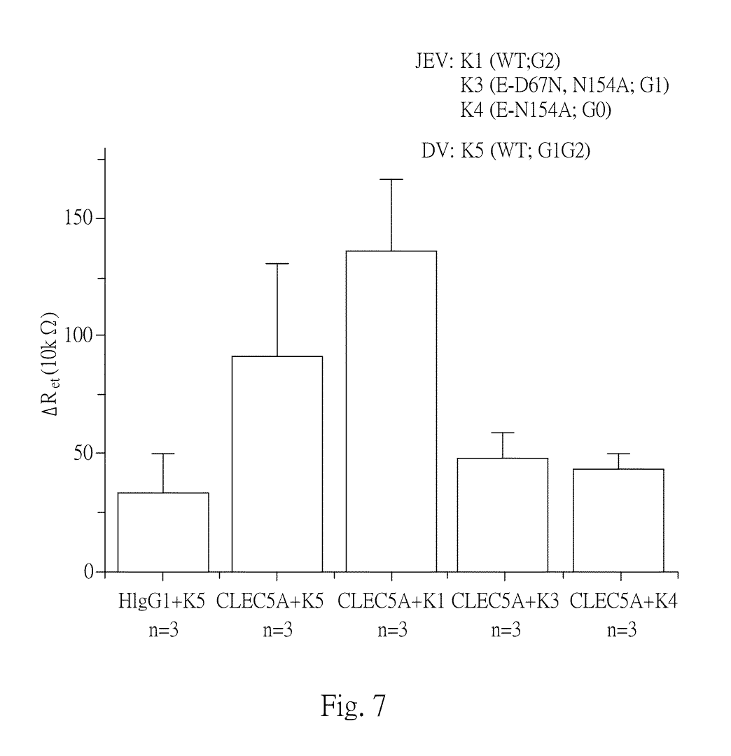

A sensor including CLEC5A as the binding domain was used to determine whether the sensor could detect differences in the glycosylation state between wild-type and mutant viruses. The flaviviruses, e.g., DV and JEV, each contain glycosylation sites in the envelope protein (E protein). DV has two such sites at positions 67 and 154, while JEV is glycosylated only at position 154. It was not known which of the two glycosylation sites were important for interacting with CLEC5A. Mutations were introduced into the JEV E protein which (i) eliminate glycosylation at position 154, (ii) add a glycosylation site at position 67, and (iii) both (i) and (ii). A sensor described above including CLEC5A as the binding domain was used as described in EXAMPLE 6 above to determine which glycosylation sites are required for JEV binding to CLEC5A. The results are shown in FIG. 7.

Both wild-type DV and JEV can be detected by a sensor including a CLEC5A binding domain. Mutation of JEV at position 154, which eliminates glycosylation at that position, results in a loss of binding to CLEC5A. See CLEC5A+K4 in FIG. 4. Similarly, a mutant of JEV having a glycosylation site at position 67 and not at position 154, i.e., mutant K3, also fails to bind to CLEC5A. Clearly, binding of JEV to CLEC5A is mediated through glycosylation at position 154 of JEV.

According the results of the examples, it suggested that the present invention can improve the sensitivity and the accuracy. Even the molecular interaction is weak, such as CLEC5A and Dengue virus, and Enterovirus 71 and P-selectin glycoprotein ligand-1, it can provide a well accuracy by the sensor of the present invention. Furthermore, it suggested that when the titer of virus is over 5×10⁶ pfu/mL and the concentration of probe is over 1 μg/well, the molecular interaction between the virus and the probe can be detected by ELISA (Chen S T et al., Nature, 2008). Even the titer of virus is much lower such as 9.5×10⁵ pfu/mL, it also can be detected by the sensor of the present invention. Compared with the prior art, the sensitivity of the present invention is increased at least 5 folds. Furthermore, compared with the prior art such as ELISA and SPR, the sensor and the detecting method of the present invention has many advantages: easier to operation, much cheaper, lower error rate and broader applications. The sensor of present invention is not only used for detecting different target, but also used for screening drug or detecting the process of the cancer cells. Moreover, it is easy and fast to calculate the concentration of the target in the sample by the present invention, so that the present invention can be the basis of treatment and lower the testing costs when it apply for disease screening or treatment.

The following documents can be used to better understand the background of the invention.

Back et al., Infect. Ecol. Epidemiol. 3:18939; Yacoub et al., Curr. Opin. Infect. Dis. 26:284-289; Miller et al., PLoS Pathogens 4:e17; Navarro-Sanchez et al., EMBO Rep. 4(7):723-728; Tassaneetrithep et al., J. Exp. Med. 197:823-829; Chen et al., Nature 453:672-676; Hsu et al., J. Biol. Chem. 284:34479-34489; Chang et al., Ann. Rev. Anal. Chem. 3:207-29; Bao et al., Anal. Bioanal. Chem. 391:933-942; Nguyen et al., Bioelectrochemistry 88:15-21; Cheng et al., Anal. Chim. Acta 725:74-80; Pingarrón et al., Electrochim. Acta 53:5848-66; Silvestrini et al., Anal. Bioanal. Chem. 405:995-1005; Yun et al., Sensors and Actuators B: Chemical 123:177-182; Tsai et al., Int. J. Nanomedicine 6:1201-1208; Chin et al., Biosens. Bioelectron. 49:506-511; Palchetti et al., Sensors and Microsystems: Springer 2010. p. 181-184; Ensafi et al., Electrochimica Acta 56:8176-8183; Weber et al., Materials Science and Engineering: C 31:821-825; Shih et al. Antimicrob. Agents Chemother. 48:3523-3529; Kumbhat et al., J. Pharm. Biomed. Anal. 52:255-259; Xu et al., Acta Veterinaria Brno 81:107-111; Heo et al., Sensors (Basel) 12:10097-10108; Diao et al., J. Electroanalytical Chemistry 495:98-105; Cheung et al., Cytometry A 65:124-132; Nishimura et al., Front. Microbiol. 3:105-109; Gunasekara et al., Biochem. Biophys. Res. Comm. 421:832-836; Watson et al., J. Biol. Chem. 286:24208-24218; and Rambaruth et al., Acta Histochem. 113:591-600.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A sensor for detecting molecular interactions between a target and a binding domain by electrochemical impedance spectroscopy, the sensor comprising:
a substrate,
an anodic aluminum oxide (AAO) barrier layer attached to the substrate, the AAO barrier layer including an array of regularly spaced nano-hemispheres spaced apart by 5-30 nm, each nano-hemisphere having a diameter of 30-300 nm,
an Au coating of 10-50 nm affixed to the AAO barrier layer,
a plurality of gold nanoparticles (GNP) having a diameter of 2-10 nm deposited on the Au coating, and
a binding domain attached to each GNP.

2. The sensor of claim 1, wherein the binding domain is covalently attached to each GNP.

3. The sensor of claim 2, wherein the binding domain is an antibody, a receptor, a recombinant protein, a glycolipid, or a glycan.

4. The sensor of claim 2, wherein the binding domain binds specifically to a virus.

5. The sensor of claim 4, wherein the virus is a herpesvirus, an adenovirus, a parvovirus, a papilloma virus, a poliovirus, an influenza virus, a rotavirus, a flavivirus, or a poxvirus.

6. The sensor of claim 4, wherein the virus is hepatitis B virus, human immunodeficiency virus, hepatitis C virus, H5N1 influenza virus, SARS virus, Japanese encephalitis virus, eastern equine encephalitis virus, West Nile virus, yellow fever virus, mumps virus, lymphocytic choriomeningitis virus, coronavirus, or Dengue virus.

7. The sensor of claim 2, wherein the binding domain is a lectin.

8. The sensor of claim 7, wherein the lectin specifically binds to a mannose residue, a fucose residue, a sialic acid residue, a glucosamine residue, or a galactosamine residue.

9. The sensor of claim 3, wherein the binding domain is a receptor-Fc fusion protein.

10. The sensor of claim 9, wherein the receptor-Fc fusion protein includes a C-type lectin domain family 5 member A (CLEC5A) binding fragment.

11. The sensor of claim 1, wherein the nano-hemispheres are arranged in staggered manners to form a restricting space among the three adjacent nano-hemispheres.

12. A method for producing an electrochemical impedance spectroscopy sensor for detecting molecular interactions, the method comprising:
forming an anodic aluminum oxide (AAO) barrier layer having an array of regularly spaced nano-hemispheres spaced apart by 5-30 nm, each nano-hemisphere having a diameter of 30-300 nm,
attaching the AAO barrier layer to a substrate,
coating the AAO barrier layer with Au,
attaching gold nanoparticles (GNPs) having a diameter of 2-10 nm to the Au-coated AAO barrier layer to form a nanostructured surface,
activating the nanostructured surface, and
attaching a binding domain to the activated nanostructured surface, thereby forming a sensor for detecting molecular interactions.

13. The method of claim 12, wherein the binding domain is selected from the group consisting of an antibody, a receptor, a recombinant protein, a glycolipid, and a glycan.

14. The method of claim 12, wherein the binding domain binds specifically to a virus.

15. The method of claim 14, wherein the virus is a herpesvirus, an adenovirus, a parvovirus, a papilloma virus, a poliovirus, an influenza virus, a rotavirus, a flavivirus, or a poxvirus.

16. The method of claim 12, wherein the binding domain binds specifically to hepatitis B virus, human immunodeficiency virus, hepatitis C virus, H5N1 influenza virus, SARS virus, Japanese encephalitis virus, eastern equine encephalitis virus, West Nile virus, yellow fever virus, mumps virus, lymphocytic choriomeningitis virus, coronavirus, or Dengue virus.

17. The method of claim 12, wherein the binding domain is a receptor-Fc fusion protein.

18. The method of claim 17, wherein the receptor-Fc fusion protein includes a C-type lectin domain family 5 member A (CLEC5A) binding fragment.

19. The method of claim 12, wherein the nano-hemispheres are arranged in staggered manners to form a restricting space among the three adjacent nano-hemispheres.

20. A method for detecting a virus in a sample, comprising providing the sensor of claim 1, measuring a first charge transfer resistance of the sensor, contacting the sensor with a sample, and measuring a second charge transfer resistance, wherein the sensor contains a binding domain that specifically binds to the virus and the second charge transfer resistance is greater than the first charge transfer resistance if the virus is present in the sample.

21. The method of claim 20, wherein the sample is a tissue sample or a blood sample.

22. The method of claim 20, wherein the binding domain is a receptor-Fc fusion protein that includes a C-type lectin domain family 5 member A (CLEC5A) binding fragment and the virus is Dengue virus or Japanese encephalitis virus.
